(19) United States
(12) Patent Application Publication
(10) Pub. No.: US 2024/0287209 A1
(43) Pub. Date: Aug. 29, 2024

(54) PROCESS FOR PREPARING POLYSACCHARIDE SULFATES, AND POLYSACCHARIDE SULFATE

(71) Applicant: FRAUNHOFER-GESELLSCHAFT ZUR FÖRDERUNG DER ANGEWANDTEN FORSCHUNG E.V., München (DE)

(72) Inventor: Kay HETTRICH, Golm (DE)

(73) Assignee: FRAUNHOFER-GESELLSCHAFT ZUR FÖRDERUNG DER ANGEWANDTEN FORSCHUNG E.V., München (DE)

(21) Appl. No.: 18/570,416

(22) PCT Filed: Jun. 18, 2021
(86) PCT No.: PCT/EP2021/066640

§ 371 Date: Dec. 14, 2023

(51) Int. Cl. C08B 5/14; A61K 9/50; B01J 13/14

(52) U.S. Cl. CPC C08B 5/14; A61K 9/5036; B01J 13/14

(57) ABSTRACT

The present invention relates to a process for preparing polysaccharide sulfates. In the process, a mixture comprising at least one polysaccharide and at least one polar aprotic solvent is provided. The at least one polysaccharide is reacted to form at least one polysaccharide acetate sulfate, wherein at least one sulphating agent, at least one acetylation agent and at least one peroxodisulfate are added to the mixture, the mixture being subsequently subjected to a temperature treatment. The at least one polysaccharide acetate sulfate is separated from the mixture and is reacted to form at least one polysaccharide sulfate. The present invention also relates to a polysaccharide sulfate that can be prepared with the process according to the invention. The present invention further relates to a microcapsule and to a process for preparing a microcapsule.

The present invention relates to a method of preparing polysaccharide sulfates. A mixture comprising at least one polysaccharide and at least one polar aprotic solvent is prepared in the method. The at least one polysaccharide is converted into at least one polysaccharide acetate sulfate in that at least one sulfating agent, at least one acetylation agent, and at least one peroxydisulfate are added to the mixture and the mixture is subsequently subjected to a temperature treatment. The at least one polysaccharide acetate sulfate is separated from the mixture and is converted into a polysaccharide sulfate. The present invention moreover relates to a polysaccharide sulfate that can be prepared using the method in accordance with the invention. The present invention furthermore also relates to a microcapsule and to a method of producing a microcapsule.

Sodium cellulose sulfate is a water-soluble polymer of the sulfuric acid half ester of the cellulose. Cationic polymers such as poly(diallyldimethylammonium chloride) (poly(DADMAC), corresponding polyelectrolyte complexes, can be formed with the aid of a watery solution of sodium cellulose sulfate by adding drops to a watery solution. Materials such as dyestuffs, flavors, but also biological objects such as cells, enzymes, bacteria can thereby be encapsulated. Sodium cellulose acetate can be formed by the esterification of the hydroxyl groups of the cellulose with a sulfating agent such as sulfuric acid anhydride, sulfuric acid, or their derivatives, and the subsequent conversion of the azide half ester into a neutral sodium salt.

Methods of preparing sodium cellulose sulfate are generally known in which the sulfating is carried out in a heterogeneous phase without dissolving the polymer (heterogeneous) or in an homogeneous phase either wile dissolving the polymer (semi-homogeneous) or after a prior dissolving of the polymer (homogeneous).

Lukanoff et al. (Lukanoff, B. and Dautzenberg, H., Das Papier, 1994, 6, 287-298) further developed a known heterogeneous preparation method (U.S. Pat. No. 2,539,451/U.S. Pat. No. 2,969,355) using sulfuric acid and propanol as the reaction medium and sulfating agent. The reaction medium is first prepared from 96% sulfuric acid and isopropanol in a molar ratio of 1.8:1 for such a heterogeneous preparation method, e.g., in accordance with Bohlmann et al. (Chemie Ingenieur Technik, 2021, 74, 359-363). The sulfating of the cellulose takes place herein at −5° C. over a time of 150 min. The reaction mixture is separated from the formed cellulose sulfuric acid half-ester and washed using alcohol to abort the reaction. The washed product is subsequently converted into the sodium salt using a sodium lye.

Substantial disadvantages of this heterogeneous sulfating process of cellulose comprise it being an exothermal reaction in a heterogeneous phase that is difficult to control and that necessarily results in irregularities in the substitute distribution along and between the polymer chains and thus impairs the solubility behavior of the obtained cellulose sulfates.

A further serious disadvantage of the heterogeneous preparation method is the fast and strong chain length reduction of the cellulose during the progressing sulfating. To diminish the chain length reduction of the cellulose, the sulfating reaction is aborted, e.g., by washing steps that remove sufficient heat and thus avoid a further temperature increase. Diffusion and expansion processes as well as the morphological structure of the cellulose nevertheless acquire a substantial influence on the reaction procedure since he reaction runs while maintaining a solid body structure of the cellulose overall.

To achieve complete water solubility of the heterogeneous prepared cellulose sulfate without separating insoluble portions in the DS range <0.8, a preactivation of the cellulose is proposed in DE 4019116 A1, with, however, only products of very low viscosity having a maximum of 8.5 mPas in a 1% solution nevertheless being obtained. When using these cellulose sulfates to produce symplex microcapsules, it must be observed that only microcapsules having a very small mechanical strength are produced.

In accordance with DE 4021049, cellulose sulfates of a higher viscosity can be isolated from the incident reaction product in that the portions insoluble in water are separated by additional method steps and the obtained soluble portions, however, have low viscosity, are washed out (cf. Lukanoff, B. and Dautzenberg, H., Das Papier, 1994, 6, 287-298).

As a result, the heterogeneous preparation process results in products with a relatively high degree of substitution (at least DS=0.7) of an inhomogeneous substitute distribution resulting therefrom and in sodium cellulose sulfate of low viscosity despite a use of high molecular starting cellulose on a conversion of the cellulose up to complete water solubility.

An intermediate cellulose derivative soluble in organic solvents is conventionally used in the homogeneous sulfating of cellulose, whereby the chain length reduction of the cellulose during the sulfating reaction can be better suppressed. Since the sulfating runs after or during a complete dissolving of the solid body structure in a dipolar aprotic solvent, a more uniform substitute distribution is achieved. The end product has a higher solution viscosity and is in part already completely water-soluble at DS values of 0.25.

Solution viscosities of the synthesized sodium cellulose sulfate up to almost 10 mPas (measurement of a 2% solution in 2N NaOh in an Ubbelohde type viscometer) are obtained, for example, on the use of relatively low molecular cellulose acetate (DS=2.4; Cuoxam-DP approximately 250 (cf. DE 4435180).

The degrees of polymerization, that are too low, of the used commercial cellulose acetates (Cuoxam-DP approximately 200 to 350) so that no cellulose sulfates of a higher solution viscosity than approximately 10 mPas in a 1% water solution can be prepared therefrom are substantial disadvantages. The setting of a corresponding solution viscosity range of the obtained sodium cellulose sulfates with a given degree of starting polymerization of the cellulose acetate is still desirable.

The acetosulfating of native cellulose as a fundamental principle for the preparation of cellulose acetate sulfate, cellulose acetate, or cellulose sulfate by mixing esterification has long been known. In this respect, almost exclusively sulfuric acids having acetic acid anhydride in glacial acetic acid as the reaction medium were used as the reactants (see e.g., U.S. Pat. No. 2,683,143). Sodium chloride sulfonate has also been used instead of sulfuric acid (U.S. Pat. No. 2,969,355). The result of the studies of Chauvelon et al. (G. Chauvelon, Carbohydrate Research, 2003, 338, 743-750) on the preparation of water-soluble cellulose acetate sulfates was a high irregularity of this heterogeneous reaction so that the target product was only able to be acquired by fractionation.

It is furthermore known that an acetosulfating of cellulose running while being dissolved is possible on a use of N,N-dimethylformamide as the reaction medium. In this respect, acetic hydride/SO₃, or acetic anhydride/chlorosulfuric acid are used as the reaction mixture (Wagenknecht et al., Das Papier, 1996, 50, 12, 712-720). After the alkaline splitting off of the unstable acetyl groups, substituted water-soluble cellulose sulfates were obtained up to DS values of approximately 0.8 exclusively in the C6 position of the anhydroglucose unit.

Disadvantages of the cellulose sulfates previously synthesized in this manner comprise the irregularity at DS<0.6 that results in heterogeneities in a watery solution and thus in unusability for the manufacture of symplex membranes or stabile polyelectrolyte complexes.

A further possibility of preparing cellulose sulfate by acetosulfating is described in EP 1863851. The chain length reduction on the precipitation is prevented by correspondingly defined neutralization conditions; the degree of polymerization and, associated therewith, the solution viscosity of the cellulose sulfate obtained after preparation are fixed.

The preparation of cellulose sulfate after solution in ionic liquids such as 1-ethyl-3-methylimidazolium acetate (EMIMAC) or 1-butyl-3-methylimidazolium chloride (BMIMCI) is described in DE 10 2007 035 322. As a result of the high viscosity, the invention makes the addition of Co solvents such as N,N-dimethylformamide (DMF) necessary. The use of ionic liquids can be named as a disadvantage in addition to this increased preparatory effort. A use of the cellulose sulfates for medical and pharmaceutical applications is only possible after a complex cleaning process due to the use of the ionic liquids. In addition, the use of ionic liquids in a large technical scale is limited by their high manufacturing costs.

Starting from this, it was the object of the present invention to provide a method by which polysaccharide sulfates can be prepared that are suitable for the production of microcapsules. It was furthermore the object of the present invention to provide a method of producing corresponding microcapsules.

This object is achieved with respect to a method of preparing polysaccharide sulfates by the features of claim 1, with respect to a polysaccharide sulfate by the features of claim 11, with respect to a method of producing microcapsules by the features of claim 14, and with respect to a microcapsule by the features of claim 16. The dependent claims represent advantageous further developments.

In accordance with the invention, a method of preparing polysaccharide sulfates is thus provided in which
a) a mixture comprising at least one polysaccharide and at least one polar aprotic solvent is prepared,
b) the at least one polysaccharide is converted into a polysaccharide acetate sulfate by adding at least one sulfating agent, at least one acetylation agent, and at least one peroxydisulfate to the mixture and subsequently subjecting the mixture to a temperature treatment,
c) the at least one polysaccharide acetate sulfate is separated from the mixture, and
d) the at least one polysaccharide acetate sulfate is converted into at least one polysaccharide sulfate.

In step a) of the method in accordance with the invention, a mixture is first prepared that comprises at least one polysaccharide such as cellulose and at least one polar aprotic solvent such as dimethylformamide. The mixture can be a dispersion. The mixture can, for example, be prepared in that the at least one polysaccharide is dispersed in the at least one polar aprotic solvent.

In step b), the at least one polysaccharide is converted into a polysaccharide acetate sulfate in that at least one sulfating agent, at least one acetylation agent, and at least one peroxydisulfate are added to the mixture (prepared in step a)) and the mixture is subsequently subjected to a temperature treatment. The at least one sulfating agent and the at least one acetylation agent are here preferably first added to the mixture and the at least one peroxydisulfate is then added to the mixture. The temperature treatment can, for example, take place at a temperature in the range from −10° C. to 150° C. for a duration of 1 min to 30 h. The at least one polysaccharide acetate sulfate can be present in dissolved form in the mixture.

In step c), the at least one polysaccharide acetate sulfate (prepared in step b)) is separated from the mixture. This can take place, for example, in that the at least one polysaccharide acetate sulfate is precipitated by adding the mixture to a precipitation medium (e.g., containing at least an alcohol and water) and the precipitated at least one polysaccharide acetate sulfate is then separated (from the mixture and the precipitation medium) by a mechanical separation process, e.g., by filtration.

In step d), the at least one polysaccharide acetate sulfate is converted into at least one polysaccharide sulfate. This can take place, for example, by alkaline splitting off of the acetate groups.

Polysaccharide sulfates can be prepared using the method in accordance with the invention that are particularly well suited for the production of microcapsules, in particular for the production of microcapsules by means of dropletization in which the shell comprises a polyelectrolyte complex of a cationic polymer such as poly-(DADMAC) and the polysaccharide sulfate. A material to be encapsulated such as an active pharmaceutical ingredient can be encapsulated in such microcapsules. As a result, such microcapsules can, for example, be used as drugs, in processes of implantation, and in processes of injection.

The method in accordance with the invention is in particular characterized by the use of at least one peroxydisulfate. It was surprisingly found that a significant increase in the degree of substitution and thus a better solubility of the prepared polysaccharide sulfate in water can be achieved by the addition of peroxdisulfate in the acetosulfation of polysaccharides, with the use of strong sulfating agents such as chlorosulfuric acid simultaneously being able to be considerably reduced. This is also advantageous since the use of strong sulfating agents, in particular of larger portions thereof, can result in a reduction of the polysaccharide chain. An increase of the degree of substitution can thus be achieved by the use of the at least one peroxydisulfate without the risk of a length reduction of the polysaccharide chain being increased. The polysaccharide sulfate prepared using the method in accordance with the invention is particularly well suited for the production of microcapsules due to the increased degree of substitution and the improved solubility in water accompanying it. These advantages can, in contrast, not be achieved by the use of sulfates such as K₂SO₄ or Na₂SO₄ (instead of the peroxydisulfates).

Peroxydisulfates are salts of the peroxydisulfuric acid that are technically used as bleaching agents and oxidation agents, but also for the initiating of the polymerization of different alkenes, including styrene, acrylonitrile, and fluoroalkenes. The polymerization is initiated by the homolysis of the peroxydisulfate. It is also known that sodium peroxydisulfate can be used for the restoration of soil and groundwater and for etching copper on printed circuit boards. Potassium and ammonium compounds are frequently used peroxydisulfates.

In the method in accordance with the invention, a so-called polysaccharide acetate sulfate, e.g., cellulose acetate sulfate, is formed during the synthesis. This mixed ester is, unlike a pure polysaccharide such as cellulose, soluble in aprotic solvents such as DMF. The synthesis used in the method in accordance with the invention is thus a quasihomogeneous synthesis, which means that a dissolving of the polysaccharide in the solvent takes place during the synthesis in that a modification of the polysaccharide into a derivative takes place that, unlike the polysaccharide, is soluble in the solvent. The solubility of the polysaccharide acetate sulfate results in a homogeneous distribution of the substitutes along the polymer chain. Such a homogeneous distribution is helpful in dissolving processes. A polysaccharide sulfate obtained by means of quasihomogeneous synthesis thus has improved solubility due to the homogeneous substitute distribution.

On the heterogeneous synthesis (that is cellulose+solvent+reactants=two phase), frequently used in the prior art, in contrast, an inhomogeneous distribution of the substitutes is typically obtained in the anhydroglucose unit (AGU) (or the anhydro monosaccharide unit or sugar unit) and along the polysaccharide chain. There is, for example, an inhomogeneous substitution at position 2, position 3, and/or position 6 in an heterogeneous synthesis of cellulose sulfate with cellulose and sulfuric acid in the AGU. It may furthermore occur that some AGUs are substituted twice or even three times and other AGUs not at all along the polymer chain. Such a product could then consequently admittedly have a total degree of substitution DS of e.g., 0.7, but could simultaneously have regions where the DS is considerably higher and other regions where the DS is considerably lower. Such products consequently have considerably worse properties such as a worse solubility in water and are therefore less well suited for the production of microcapsules.

In the homogeneous synthesis, in which a dissolving of the polysaccharide in a solvent takes place prior to the synthesis, and in the quasihomogeneous synthesis, in which a dissolving of the polysaccharide takes place by modification into a derivative during the synthesis, there is typically a homogeneous distribution of the substitutes along the polymer chain and frequently a regioselective substitution within the AGU (pr the anydromonosaccharide unit) in the prior art. Substitution thus often first takes place primarily at the C6 position in acetosulfation.

In the method in accordance with the invention that is based on a quasihomogeneous synthesis, in contrast, a different regioselective substitute distribution is obtained within an AGU (or anhydro monosaccharide unit). A substitution can thus, for example, take place not only primarily at the C6 position, but also to larger extents at the C2 position so that a more homogeneous distribution of the substitutes within an AGU (or anhydro monosaccharide unit) is also obtained in addition to the homogeneous distribution of the substitutes along the polymer chain. It has surprisingly been found that the specific regioselective substitute distribution due to the use of the peroxydisulfate and the more homogeneous distribution of the substitutes within an AGU (or anhydro monosaccharide unit) resulting therefrom together with the homogeneous distribution of the substitutes along the polymer chain results in an even better solubility of the prepared polysaccharide acetate sulfate in water. The polysaccharide sulfates prepared using the method in accordance with the invention are also particularly well suited for the production of microcapsules for this reason.

Viewed overall, the polysaccharide sulfates prepared using the method in accordance with the invention have a higher degree of substitution, a homogeneous substitute distribution, and an advantageous regioselective substitute distribution (within an AGU or anhydro monosaccharide unit) due to the specific preparation. These advantageous properties result in a very good solubility of the prepared polysaccharide sulfate in water so that the polysaccharide sulfates prepared using the method in accordance with the invention are particularly well suited for the production of microcapsules.

A preferred variant of the method in accordance with the invention is characterized in that the at least one polysaccharide is selected from the group consisting of cellulose, hemicellulose, chitosan, hyaluronic acid, hydroxyethyl cellulose, hydroxypropyl cellulose, methylhydroxyethyl cellulose, methylhydroxypropyl cellulose, methylhydroxybutyl cellulose, ethylhydroxyethyl cellulose, carboxymethylhydroxyethyl cellulose and mixtures thereof. The at least one polysaccharide is particularly preferably cellulose.

In accordance with a further preferred variant of the method in accordance with the invention, the at least one polar aprotic solvent is selected from the group consisting of
tertiary carboxylic acid amides, e.g., dimethylformamide,
carbonic acid esters, e.g., dimethylcarbonate,
sulfoxides, e.g., dimethyl sulfoxide,
lactams, e.g., N-methyl-2-pyrrolidone, and
mixtures thereof.

A further preferred variant of the method in accordance with the invention is characterized in that the mixture in step a) is prepared in that the at least one polysaccharide is dispersed in the at least one polar aprotic solvent. The mixture (or dispersion) thus obtained is preferably stirred prior to step b) at a temperature in the range from 10° C. to 150° C., preferably from 50° C. to 120° C., and/or for a duration of 1 min to 10 h, preferably of 30 min to 5 h.

A further preferred variant of the method in accordance with the invention is characterized in that
the at least one sulfating agent is selected from the group consisting of sulfuric acid, chlorosulfuric acid, SO₃ complexes, sulfamic acid, sulfuryl chloride, and mixtures thereof, and/or
the at least one acetylation agent is selected from the group consisting of acetic acid anhydride, acetyl chloride, and mixtures thereof, and/or
the at least one peroxydisulfate is selected from the group consisting of potassium peroxydisulfate, ammonium peroxydisulfate, sodium peroxydisulfate, and mixtures thereof.

In accordance with a further preferred variant of the method in accordance with the invention, the mixture prepared in step a) contains a maximum of 3 mol/mol AGU (or anhydro monosaccharide unit), preferably a maximum of 2 mol/mol AGU (or anhydro monosaccharide unit), particularly preferably a maximum of 1 mol/mol AGU (or anhydro monosaccharide unit), and very particularly preferably a maximum of 0.5 mol/mol AGU (or anhydro monosaccharide unit) of the at least one sulfating agent.

A further preferred variant of the method in accordance with the invention is characterized in that in step b), the at least one sulfating agent and the at least one acetylation agent are first added to the mixture and the at least one peroxydisulfate is then added to the mixture.

A further preferred variant of the process in accordance with the invention is characterized in that the temperature treatment in step b) takes place
at a temperature in the range from −10° C. to 150° C., preferably from 30° C. to 100° C., particularly preferably from 45° C. to 80° C., and/or
for a time period of 1 min to 30 h, preferably of 30 min to 20 h, particularly preferably of 3 h to 10 h.

In accordance with a further preferred variant of the method in accordance with the invention, in step c), the at least one polysaccharide acetate sulfate is separated from the mixture in that the at least one polysaccharide acetate sulfate is precipitated by adding the mixture to a precipitation medium containing at least an alcohol and water and is then separated by a mechanical separation process, preferably by filtration. The at least one polysaccharide acetate sulfate is preferably washed using a washing solution once or several times after the separation.

A further preferred variant of the method in accordance with the invention is characterized in that in step d), the at least one polysaccharide acetate sulfate is converted into the at least one polysaccharide sulfate by alkaline splitting off of the acetate groups. The alkaline splitting off of the acetate groups is preferably achieved in that the at least one polysaccharide acetate sulfate is admixed with an alkaline solution and the mixture thus produced is stirred for a time period of 1 min to 30 h, preferably of 1 h to 20 h, particularly preferably of 5 h to 15 h. It is preferred that the mixture is neutralized after the stirring and the at least one polysaccharide is separated, is washed once or several times, and is dried. The present invention furthermore relates to a polysaccharide sulfate that can be or is prepared using the method in accordance with the invention.

The polysaccharide sulfate in accordance with the invention has a specific regioselective substitute distribution within the individual AGUs (or anhydro monosaccharide units) by which the polysaccharide sulfate in accordance with the invention differs from already known polysaccharide sulfate due to the method in accordance with the invention, in particular due to the use of the peroxydisulfate. The exact substitute distribution is also dependent to a certain degree on the polysaccharide respectively used in the preparation so that no general substitute distribution can be given that applies to all polysaccharide sulfates. As a result, the polysaccharide sulfate in accordance with the invention is characterized via the preparation process.

In polysaccharide chemistry, the degree of substitution indicates how many OH groups are substituted in the sugar unit (or anhydro monosaccharide unit). In the case of cellulose, the DS value can be a maximum of 3 with 3 OH groups in the glucose unit (or AGU). As a rule and in dependence on the determination method, the degree of substitution is given as a sum parameter such as in the determination of heteroatoms such as sulfur and nitrogen by elemental analysis. In specific spectroscopic methods such as ¹³C-NMR spectroscopy, an association of the regioselectivity in the structural unit is possible under certain circumstances. It can thus be possible to determine the substitution at positions C6, C2, and C3.

With polysaccharide sulfates, the degrees of substitution of the individual C positions, e.g., the degree of substitution DS₂ at the C2 position or the degree of substitution DS₆ at the C6 position of the polysaccharide sulfate can be determined by means of ¹³C-NMR spectroscopy. The measurement of the NMR spectrum can take place here e.g., in D₂O at 60° C. The substitution can be quantified by integrating the signals from the ¹³C-NMR spectrum and standardizing to a signal of a C atom, e.g., C1. Such a procedure is described in e.g., Zhant et al.: “Synthesis and spectroscopic analysis of cellulose sulfates with regulable total degrees of substitution and sulfation patterns via ¹³C NMR and FT Raman spectroscopy”, Polymer, 52 (1), pages 26-32.

A preferred embodiment of the polysaccharide sulfate in accordance with the invention is characterized in that the polysaccharide sulfate
has a solution viscosity of at least 0.5 mm²/s, preferably of at least 2 mm²/s, in a 1% solution in water, and/or
has a (total) degree of substitution DS in a range of 0.15 to 1.8, preferably of 0.5 to 1.3 (e.g., determined via the sulfur content of the polysaccharide sulfate determined by means of elemental analysis or via ¹³C-NMR spectroscopy).

The solution viscosity can be determined, for example, by means of DIN 51562-1: 1999-01.

The degree of substitution DS or the total degree of substitution DS indicates the proportion at which a substitution (of a hydroxyl group for a sulfate group) can take place at the C positions, i.e., at which there is a hydroxyl group in the original polysaccharide, also at which a substitution (of the original hydroxyl group for a sulfate group) has actually taken place. The (total) degree of substitution DS can adopt a value in the range from 0 to z, where z there corresponds to the number of C positions in the anhydro glucose unit of the polysaccharide at which a substitution (of a hydroxyl group for a sulfate group) can take place, i.e., at which there is a hydroxyl group in the original polysaccharide. The anhydro glucose unit of cellulose, for example, contains three C positions at which a substitution (of a hydroxyl group for a sulfate group) can take place, namely the C2 position, the C3 position, and the C6 position. The cellulose sulfate of the (total) degree of substitution DS can consequently adopt a value in the range from 0 to 3, wherein, at the minimum value of 0, a substitution has taken place at no position and, at the maximum value of 3, a substitution has taken place at every C2, C3, and C6 position in the polysaccharide. For example, a value of 1.5 for the (total) degree of substitution DS of cellulose sulfate would mean that a substitution (of the original hydroxyl group for a sulfate group) has taken place at 50% or half of all the possible substitution positions (i.e., the sum of all C2, C3, and C6 positions) of the polysaccharide sulfate. The (total) degree of substitution DS here does not allow any direct conclusion to be drawn on how high the degree of substitution is at the individual C positions. A value of 1.5 for the (total) degree of substitution DS of cellulose sulfate can mean, for example, that a substitution (of a hydroxyl group by a sulfate group) has taken place at all C6 positions, at half the C2 positions and at none of the C3 positions.

Alternatively, a value of 1.5 for the (total) degree of substitution DS of cellulose sulfate can, however, also mean, for example, that a substitution (of a hydroxyl group by a sulfate group) has taken place at half the C6 positions, at half the C2 positions, and at half the C3 positions.

The degree of substitution DS or the total degree of substitution DS can be determined via the sulfur content of the polysaccharide sulfate, with the determination of the sulfur content of the polysaccharide sulfate being able to take place by means of elemental analysis. The determination of the degree of substitution via the sulfur content can take place using the following formula (A):

$$DS = ( {M}_{PS} \times S [ \% ] ) / ( 100 \times {M}_{S} - Δ M \times S [ \% ] ) \qquad Formula ( A )$$

where M_{S} is the molar mass of the element to be determined, of the sulfur in this case, M_{PS} is the molar mass of the polysaccharide used, and ΔM is the difference between the molar mass of the new substitute (e.g., SO₃) and the leaving group (e.g., H). Such a determination of the degree of substitution is also described, for example, in Rohowsky et al., Carbohydr. Polymers, 2016, 142, 56-62.

Alternatively, the degree of substitution DS or the total degree of substitution DS can also be determined by means of ¹³C-NMR spectroscopy. The measurement of the NMR spectrum can take place here e.g., in D₂O at 60° C. The determination of the degree of substitution from the ¹³C-NMR spectrum can then take place by integrating the signals from the ¹³C-NMR spectrum and standardizing to a signal of a C atom, e.g., C1 (see e.g., Zhang et al., Polymer, 52(1), pp. 26-32). The substitution at the individual C atoms in the AGU (or the anhydro monosaccharide unit) can also be determined by ¹³C-NMR spectroscopy.

A further preferred embodiment of the polysaccharide sulfate in accordance with the invention is characterized in that the polysaccharide sulfate has a degree of substitution DS₂ at the C2 position of at least 0.2, preferably at least 0.3, particularly preferably at least 0.4 and/or has a degree of substitution DS₆ at the C6 position of at most 0.9, preferably at most 0.8, particularly preferably at most 0.7, and very particularly preferably at most 0.6.

The degree of substitution of the individual C positions, e.g., the degree of substitution DS₂ at the C2 position and the degree of substitution DS₆ at the C6 position of the polysaccharide sulfate can be determined by means of ¹³C-NMR spectroscopy. The measurement of the NMR spectrum can take place here e.g., in D₂O at 60° C. The determination of the individual degrees of substitution from the ¹³C-NMR spectrum can then take place by integrating the signals from the ¹³C-NMR spectrum and standardizing to a signal of a C atom, e.g., C1 (see e.g., Zhang et al., Polymer, 52(1), pp. 26-32).

A very particularly preferred embodiment of the polysaccharide sulfate in accordance with the invention is characterized in that the polysaccharide sulfate is cellulose sulfate, the cellulose sulfate having a degree of substitution DS₂ at the C2 position of at least 0.2, preferably at least 0.3, particularly preferably at least 0.4 and/or having a degree of substitution DS₆ at the C6 position of at most 0.9, preferably at most 0.8, particularly preferably at most 0.7, and very particularly preferably at most 0.6.

The present invention also relates to a polysaccharide sulfate (preferably cellulose sulfate) that has a degree of substitution DS₂ at the C2 position of at least 0.2, preferably at least 0.3, particularly preferably at least 0.4 and/or has a degree of substitution DS₆ at the C6 position of at most 0.9, preferably at most 0.8, particularly preferably at most 0.7, and very particularly preferably at most 0.6.

The present invention furthermore relates to a method of producing microcapsules in which
at least one polysaccharide sulfate is prepared using the method in accordance with the invention for preparing polysaccharide sulfate, or
at least one polysaccharide sulfate in accordance with the invention is provided,
   and then
e) an aqeuous solution of the at least one polysaccharide sulfate is prepared,
f) at least one material to be encapsulated is added to the aqueous solution of the at least one polysaccharide sulfate, whereby a suspension is produced,
g) a dropletization of at least some of the suspension is carried out, whereby drops of the suspension are produced, and
h) the drops of the suspension are dropped into a solution of at least one cationic polymer, with the cationic polymer forming a polyelectrolyte complex with the polysaccharide sulfate and the drops thereby being converted into microcapsules in which the material to be encapsulated is encapsulated.

A preferred variant of the method in accordance with the invention for producing microcapsules is characterized in that in the method
a) a mixture comprising at least one polysaccharide and at least one polar aprotic solvent is prepared,
b) the at least one polysaccharide is converted into a polysaccharide acetate sulfate by adding at least one sulfating agent, at least one acetylation agent, and at least one peroxydisulfate to the mixture and subsequently subjecting the mixture to a temperature treatment,
c) the at least one polysaccharide acetate sulfate is separated from the mixture,
d) the at least one polysaccharide acetate sulfate is converted into at least one polysaccharide sulfate.
e) an aqeuous solution of the at least one polysaccharide sulfate is prepared,
f) at least one material to be encapsulated is added to the aqueous solution of the at least one polysaccharide sulfate, whereby a suspension is produced,
g) a dropletization of at least some of the suspension is carried out, whereby drops of the suspension are produced, and
h) the drops of the suspension are dropped into a solution of at least one cationic polymer, with the cationic polymer forming a polyelectrolyte complex with the polysaccharide sulfate and the drops thereby being converted into microcapsules in which the material to be encapsulated is encapsulated.

The produced microcapsules preferably have a diameter of 0.1 μm to 1,000,000 μm. particularly of 1 μm to 10000 μm, very particularly preferably of 10 μm to 1000 μm.

A further preferred variant of the method in accordance with the invention is characterized in that
the aqueous solution of the at least one polysaccharide sulfate prepared in step e) is a 0.5% to 10% solution of the at least one polysaccharide sulfate in water, and/or
the at least one material to be encapsulated is a material of biological origin or is a material of non-biological origin, and/or
in step f), one or more substances selected from the group consisting of carrier materials, additives, solvents, e.g., DMSO, preservatives, salts, glycerin, and mixtures thereof is/are additionally added to the aqueous solution of the at least one polysaccharide, and/or
the at least one cationic polymer is selected from the group consisting of polyethylenediamine, polypiperazine, polyarginine, polytriethylamine, spermine, polydimethylallylammonium, polydiallyldimethylammonium, polyvinylbenzyltrimethylammonium, cationic chitosans, derivatives of cationic chitosans, and mixtures thereof, and/or
the solution of the at least one cationic polymer is an aqueous solution of the at least one cationic polymer.

The at least one material to be encapsulated can be at least one material of biological origin. Alternatively, the at least one material to be encapsulated can be at least one material of non-biological origin. For example, the at least one material to be encapsulated can be at least one active pharmaceutical ingredient. For example, the at least one material to be encapsulated can be at least one substance that is used as a drug. The active pharmaceutical ingredient or the drug can be implanted or injected encapsulated in the microcapsule.

Alternatively, the at least one material to be encapsulated can be at least one substance that is not an active pharmaceutical ingredient and not a drug.

The present invention furthermore also relates to a microcapsule comprising at least a material to be encapsulated and a shell surrounding the at least one material to be encapsulated, with the shell containing a polyelectrolyte complex of at least one cationic polymer and at least one polysaccharide sulfate in accordance with the invention.

Preferably, the microcapsule in accordance with the invention can be or is produced using the method in accordance with the invention for producing microcapsules.

The microcapsule in accordance with the invention preferably has a diameter of 0.1 μm to 1,000,000 μm. particularly preferably of 1 μm to 10000 μm, very particularly preferably of 10 μm to 1000 μm.

The present invention also relates to a microcapsule in accordance with the invention for use as a drug, for use in a process of implantation, or for use in a process of injection.

The present invention further relates to the use of the microcapsule in accordance with the invention as a drug, in a process of implantation or in a process of injection.

The present invention is explained based on the following figures and examples in more detail without restricting the invention to the parameters specifically shown.

### EMBODIMENT 1

5 g (atro) of a cellulose (cotton linter) are dispersed in 150 ml N,N-dimethylformamide (DMF) and are stirred at 85° C. for 2 hours.

The sulfating was started by the addition of 4 mL chlorosulfuric acid (1 mol/mol AGU)+70 mL acetic acid anhydride (12 mol/mol AGU) in 80 mL DMF. A suspension of 8.3 kg K₂S₂O₈ (0.5 mol/mol AGU) in 50 mL DMF is subsequently added. The synthesis took place at a temperature of 65° C. The polymer dissolves in the solvent after 1 to 2 hours.

The precipitation took place after 5 hours while stirring continuously by a slow pouring of the polymer solution (within 10 min) into a room temperature precipitation medium that was composed of 21 g sodium hydroxide (NaOH), 42 g H₂O, and 10 g sodium acetate, filled to 750 mL ethanol. Stirring continued for 1 hour after the end of precipitation. Filtering subsequently took place and washing three times with respectively 300 ml of a washing solution consisting of 4% (w/w) sodium acetate in an ethanol-water mixture (1:1, w/w). The polymer or the precipitation product was subsequently stirred for 12 h into an alkaline solution (8 g NaOH, 16 g 60, 200 mL ethanol) for splitting off the acetate groups. After neutralization with an ethanolic acetic acid (pH setting between 6 and 9), three washes followed in 300 mL ethanol respectively and the washed product was dried in a vacuum drying cupboard.

The cellulose sulfate prepared in this manner has a total degree of substitution DS of 0.8 (determined via the sulfur content of the cellulose sulfate determined by means of elemental analysis using formula (A)) and a viscosity of 14 mm²/s (determined in accordance with DIN 51562-1:1999-01). Further properties of the prepared cellulose sulfate can be seen from Table 1.

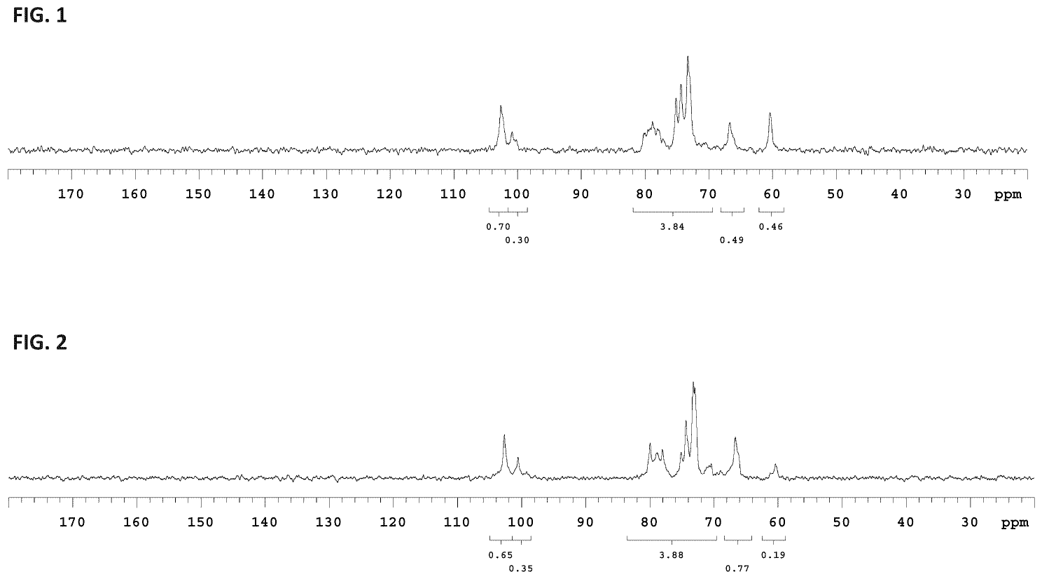

In addition, a ¹³C-NMR spectrum in D₂O of the prepared cellulose sulfate was recorded at a temperature of 60° C. The spectrum obtained is shown in FIG. 1.

A determination was able to be made from the ¹³C-NMR spectrum that the prepared cellulose acerate has a degree of substitution DS₂ at the C2 position of 0.30 and a degree of substitution DS₆ at the C6 position of 0.49. The determination took place by integrating the signals from the ¹³C-NMR spectrum and standardizing to a signal of a C atom, e.g., C1 (see e.g., Zhang et al., Polymer, 52(1), pp. 26-32). A (total) degree of substitution DS of 0.79 thus results from the ¹³C-NMR spectrum that correlates with the (total) degree of substitution of 0.8. determined within the rounding accuracy via the sulfur content.

### EMBODIMENT 2

5 g (atro) of a cellulose (cotton linter) are dispersed in 150 ml N,N-dimethylformamide (DMF) and are stirred at 85° C. for 2 hours.

The sulfating was started by the addition of 2 mL chlorosulfuric acid (0.5 mol/mol AGU)+70 mL acetic acid anhydride (12 mol/mol AGU) in 80 mL DMF. A suspension of 14 g (NH₄)₂S₂O₈ (1 mol/mol AGU in 50 mL DMF is subsequently added. The synthesis took place at a temperature of 75° C. The polymer dissolves in the solvent after approximately 1 to 2 hours.

The precipitation and preparation took place after 6 hours as described in Example 1.

The cellulose sulfate prepared in this manner has a total degree of substitution DS of 1.2 (determined via the sulfur content of the cellulose sulfate determined by means of elemental analysis using formula (A)) and a viscosity of 2 mm²/s (determined in accordance with DIN 51562-1:1999-01). Further properties of the prepared cellulose sulfate can be seen from Table 1.

In addition, a ¹³C-NMR spectrum in D₂O of the prepared cellulose sulfate was recorded at a temperature of 60° C. The spectrum obtained is shown in FIG. 2.

A determination was able to be made from the ¹³C-NMR spectrum that the prepared cellulose acerate has a degree of substitution DS₂ at the C2 position of 0.35 and a degree of substitution DS₆ at the C6 position of 0.77. The determination took place by integrating the signals from the ¹³C-NMR spectrum and standardizing to a signal of a C atom, e.g., C1 (see e.g., Zhang et al., Polymer, 52(1), pp. 26-32). A (total) degree of substitution DS of 1.12 thus results from the ¹³C-NMR spectrum that correlates with the (total) degree of substitution of 1.2. determined within the rounding accuracy via the sulfur content.

### EMBODIMENT 3

5 g (atro) of a microcrystalline cellulose (MCC) were dispersed in 150 ml DMF and were stirred at 85° C. for 3 hours.

The sulfating was started by the addition of 2.5 g sulfuric acid trioxide/pyridine complex (0.5 mol/mol AGU)+70 mL acetic acid anhydride (12 mol/mol AGU) dissolved in 50 mL DMF.

The synthesis took place at a temperature of 60° C. Subsequently a suspension of 14 g (NH₄)₂S₂O₈ (4 mol/mol AGU) in 50 mL DMF is added. The polymer dissolves in the solvent after 1 to 2 hours.

The precipitation and preparation took place after 4 hours as described in Example 1.

The cellulose sulfate prepared in this manner has a total degree of substitution DS of 0.85 (determined via the sulfur content of the cellulose sulfate determined by means of elemental analysis using formula (A)) and a viscosity of 1 mm²/s (determined in accordance with DIN 51562-1:1999-01). Further properties of the prepared cellulose sulfate can be seen from Table 1.

### EMBODIMENT 4

5 g (atro) of a cellulose (fir pulp) were dispersed in 150 ml DMF and were stirred at 85° C. for 3 hours.

The sulfating was started by the addition of 1.2 ml sulfuric acid (0.7 mol/mol AGU)+70 mL acetic acid anhydride (12 mol/mol AGU) in 80 mL DMF. A suspension of 8.3 kg K₂S₂O₈ (0.5 mol/mol AGU) in 50 mL DMF is subsequently added. The synthesis took place at a temperature of 50° C. The polymer dissolves in the solvent after approximately 1 to 2 hours.

The precipitation and preparation took place after 8 hours as described in Example 1.

The cellulose sulfate prepared in this manner has a total degree of substitution DS of 1.0 (determined via the sulfur content of the cellulose sulfate determined by means of elemental analysis using formula (A)) and a viscosity of 10 mm²/s (determined in accordance with DIN 51562-1:1999-01). Further properties of the prepared cellulose sulfate can be seen from Table 1.

### EMBODIMENT 5

5 g (atro) of a cellulose (eucalyptus pulp) were dispersed in 150 ml DMF and were stirred at 85° C. for 3 hours.

The sulfating was started by the addition of 2 mL chlorosulfuric acid (0.5 mol/mol AGU)+70 mL acetic acid anhydride (12 mol/mol AGU) in 80 mL DMF. A suspension of 14 g (NH₄)₂S₂O₈ (4 mol/mol AGU in 50 mL DMF is subsequently added. The synthesis took place at a temperature of 75° C. The polymer dissolves in the solvent after approximately 1 to 2 hours. The precipitation and preparation took place after 6 hours as described in Example 1.

The cellulose sulfate prepared in this manner has a total degree of substitution DS of 1.3 (determined via the sulfur content of the cellulose sulfate determined by means of elemental analysis using formula (A)) and a viscosity of 22 mm²/s (determined in accordance with DIN 51562-1:1999-01). Further properties of the prepared cellulose sulfate can be seen from Table 1.

### EMBODIMENT 6

5 g (atro) of an arabinoxylan (birch) were dispersed in 150 ml DMF and were stirred at 85° C. for 3 hours.

The sulfating was started by the addition of 1.2 mL chlorosulfuric acid (0.5 mol/mol AGU)+70 mL acetic acid anhydride (12 mol/mol AGU) in 80 mL DMF. A suspension of 5.4 kg K₂S₂O₈ (0.5 mol/mol AGU) in 50 mL DMF is subsequently added. The synthesis took place at a temperature of 55° C. The polymer dissolves in the solvent after approximately 1 to 2 hours.

The precipitation and preparation took place after 6 hours as described in Example 1. The last washing steps took place with the aid of a dialysis tube, however.

The arabinoxylan sulfate prepared in this manner has a total degree of substitution DS of 0.9 (determined via the sulfur content of the arabinoxylan sulfate determined by means of elemental analysis using formula (A)) and a viscosity of 2 mm²/s (determined in accordance with DIN 51562-1:1999-01). Further properties of the prepared arabinoxylan sulfate can be seen from Table 1.

TABLE 1
Properties of the polysaccharide sulfates prepared
in accordance with Embodiments 1 to 6 (viscosity
and clouding were measured in 1% (w/w) solutions).

| Embodi- | Yield | | | Viscosity v | Clouding | Encapsu- |
|---|---|---|---|---|---|---|
| ment | [g] | DS^{S} | DS^{NMR} | [mm²/s] | [NTU] | lation |
| 1 | 3 | 0.8 | 0.79 | 14 | 8 | Yes |
| 2 | 3.5 | 1.2 | 1.12 | 2 | 8 | Yes |
| 3 | 3.5 | 0.85 | — | 1 | 6 | Yes |
| 4 | 4.2 | 1.0 | — | 10 | 7 | Yes |
| 5 | 5.4 | 1.3 | — | 22 | 9 | Yes |
| 6 | 4 | 0.9 | — | 2 | 12 | Yes, |
| | | | | | | shape- |
| | | | | | | less |

The (total) degrees of substitution DS^{S} in Table 1 were determined via the sulfur content of the cellulose sulfate determined by means of elemental analysis using formula (A). The (total) degrees of substitution DS^{NMR} in Table 1 were determined by means of ¹³C-NMR spectroscopy by integrating the signals from the ¹³C-NMR spectrum and standardizing to a signal of a C atom, e.g., C1 (see e.g., Zhang et al., Polymer, 52(1), pp. 26-32). The values for the viscosity in Table 1 were determined in accordance with DIN 51562-1:1999-01. The values for the clouding in Table 1 were determined by means of DIN EN ISO 7027-1:2016-11.

Microcapsules were able to be produced successfully with all of the polysaccharide sulfates prepared in accordance with Embodiments 1 to 6. Only shapeless microcapsules were able to be obtained by the polysaccharide sulfate obtained in Embodiment 6.

### EMBODIMENT 7

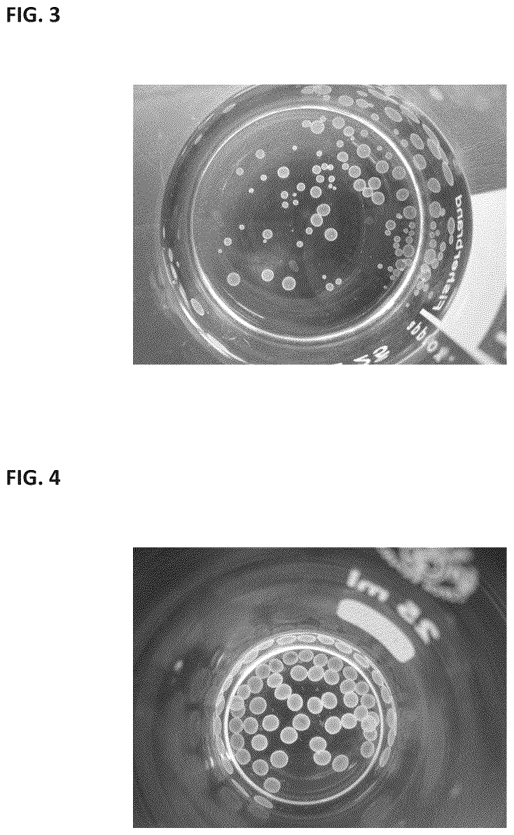

An aqueous solution (1% w/w) is prepared by a corresponding weighted portion from the cellulose sulfate prepared in Embodiment 1. After the substance has completely dissolved, a material to be encapsulated is added to the aqueous solution of the at least one polysaccharide sulfate, whereby a suspension is produced. The cellulose sulfate solution is subsequently added by dropping into a 1% commercially available polydiallyldimethylammonium chloride solution (polyDADMAC solution). Homogeneous round spherical particles (microcapsules) are obtained. The material to be encapsulated is encapsulated in the microcapsules obtained. The capsules obtained are shown in the photolithographic shots in FIG. 3 and FIG. 4.

1-18. (canceled)

19. A method of preparing at least one polysaccharide sulfate comprising:
a) preparing a mixture comprising at least one polysaccharide and at least one polar aprotic solvent,
b) converting the at least one polysaccharide into a polysaccharide acetate sulfate by adding at least one sulfating agent, at least one acetylation agent, and at least one peroxydisulfate to the mixture and subsequently subjecting the mixture to a temperature treatment,
c) separating the at least one polysaccharide acetate sulfate from the mixture, and
d) converting the at least one polysaccharide acetate sulfate into at least one polysaccharide sulfate.

20. The method in accordance with claim 19, wherein the at least one polysaccharide is selected from the group consisting of cellulose, hemicellulose, chitosan, hyaluronic acid, hydroxyethyl cellulose, hydroxypropyl cellulose, methylhydroxyethyl cellulose, methylhydroxypropyl cellulose, methylhydroxybutyl cellulose, ethylhydroxyethyl cellulose, and carboxymethylhydroxyethyl cellulose thereof.

21. The method in accordance with claim 19, wherein the at least one polar aprotic solvent is selected from the group consisting of
tertiary carboxylic acid amides,
carbonic acid esters,
sulfoxides, and
lactams.

22. The method in accordance with claim 19, wherein the mixture in step a) is prepared by dispersing the at least one polysaccharide in the at least one polar aprotic solvent.

23. The method in accordance with claim 19, wherein
the at least one sulfating agent is selected from the group consisting of sulfuric acid, chlorosulfuric acid, SO₃ complexes, sulfamic acid, and sulfuryl chloride, and/or
the at least one acetylation agent is selected from the group consisting of acetic acid anhydride and acetyl chloride, and/or
the at least one peroxydisulfate is selected from the group consisting of potassium peroxydisulfate, ammonium peroxydisulfate, and sodium peroxydisulfate.

24. The method in accordance with claim 19, wherein in step b), the at least one sulfating agent and the at least one acetylation agent are first added to the mixture and the at least one peroxydisulfate is then added to the mixture.

25. The method in accordance with claim 19, wherein the temperature treatment in step b) takes place
at a temperature in the range from −10° C. to 150° C., and/or
for a time period of 1 min to 30 h.

26. The method in accordance with claim 19, wherein in step c), the at least one polysaccharide acetate sulfate is separated from the mixture by adding the mixture to a precipitation medium containing at least an alcohol and water and subjecting the resulting mixture to a mechanical separation process.

27. The method in accordance with claim 19, wherein in step d), the at least one polysaccharide acetate sulfate is converted into the at least one polysaccharide sulfate by alkaline splitting off of the acetate groups.

28. The method in accordance with claim 27, wherein the alkaline splitting off of the acetate groups is carried out by admixing the at least one polysaccharide acetate sulfate with an alkaline solution and the mixture thus produced is stirred for a time period of 1 min to 30 h.

29. The method in accordance with claim 28, further comprising neutralizing the mixture after the stirring and the at least one polysaccharide is separated, washed once or several times, and dried.

30. A polysaccharide sulfate prepared by the method of claim 19.

31. The polysaccharide sulfate in accordance with claim 30, wherein the polysaccharide sulfate
has a solution viscosity of at least 0.5 mm²/s in a 1% solution in water, and/or
has a degree of substitution DS in a range from 0.15 to 1.8.

32. The polysaccharide sulfate in accordance with claim 30, wherein the polysaccharide sulfate has a degree of substitution DS₂ at the C2 position of at least 0.2, and/or has a degree of substitution DS₆ at the C6 position of at most 0.9.

33. A method of producing microcapsules comprising preparing at least one polysaccharide sulfate prepared by the method of claim 19, and
a) preparing an aqueous solution of the at least one polysaccharide sulfate,
b) adding at least one material to be encapsulated to the aqueous solution of the at least one polysaccharide sulfate, whereby a suspension is produced,
c) carrying out dropletization of at least some of the suspension, whereby drops of the suspension are produced, and
d) dropping the drops of the suspension into a solution of a cationic polymer, with the cationic polymer forming a polyelectrolyte complex with the polysaccharide sulfate and the drops thereby being converted into microcapsules in which the material to be encapsulated is encapsulated.

34. The method in accordance with claim 33, wherein
the aqueous solution of the at least one polysaccharide sulfate prepared is a 0.5% to 10% solution of the at least one polysaccharide sulfate in water, and/or
the at least one material to be encapsulated is a material of biological origin or a material of non-biological origin.

35. The method in accordance with claim 34, wherein one or more substances selected from the group consisting of carrier materials, additives, and solvents is/are further added to the aqueous solution of the at least one polysaccharide.

36. The method in accordance with claim 33, wherein:
the at least one cationic polymer is selected from the group consisting of polyethylenediamine, polypiperazine, polyarginine, polytriethylamine, spermine, polydimethylallylammonium, polydiallyldimethylammonium, polyvinylbenzyltrimethylammonium, cationic chitosans, and derivatives of cationic chitosans, and/or
the solution of the at least one cationic polymer is an aqueous solution of the at least one cationic polymer.

37. A microcapsule comprising at least one encapsulated material and a shell surrounding the at least one encapsulated material, with the shell containing a polyelectrolyte complex of at least one cationic polymer and at least one polysaccharide sulfate in accordance with claim 33.

38. A drug formulation, injection formulation, or an implantation comprising a microcapsule according to claim 37.
